SEQUENCE LISTING (23 sequences)

SEQ ID NO: 1 - DNA, 1866 bp, unidentified
  source 1..1866: mol_type = other DNA; note = artificial sequence
atgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 2 - DNA, 1194 bp, unidentified
  source 1..1194: mol_type = other DNA; note = artificial sequence
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 3 - DNA, 2222 bp, unidentified
  source 1..2222: mol_type = other DNA; note = artificial sequence
aataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcgggatcctcaggtagtaagtattcattgtaaatctgatattatttgtattattatacctacctaatttgcagtgcagatcattgagataattaaaattataaccatctcgcaaataaataagtattttactgttttcgtaacagttttgtaataaaaaaacctataaatcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 4 - DNA, 2583 bp, unidentified
  source 1..2583: mol_type = other DNA; note = artificial sequence
gacctttaattcaacccaacacaatatattatagttaaataagaattattatcaaatcatttgtatattaattaaaatactatactgtaaattacattttatttacaatcactcgacgaagacttgatcacccgggggacaaggtaagtattcattgtaaatctgatattatttgtattattatacctacctaatttgcagtgcaggccgccatggtaagtatcgataactttgttttctttcacatttacaactccgacatacaaattgtaattttattactgtttggtccataaacacttgtttaccatttccttttttacaagttttaatattttctgcatatataaaacattatttattttgcaggctgccgacggttatctacccgattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 5 - DNA, 2407 bp, unidentified
  source 1..2407: mol_type = other DNA; note = artificial sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgccgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 2406 bp, unidentified
  source 1..2406: mol_type = other DNA; note = artificial sequence
cacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaacattgtaaacgtgccggagcaacaatctccggagactgcggccgtgtgcaaaaattaaaagctgttgaataaactggaatcgagctcttacaacaaatccaacattgaccagctggccgttattgtaaaggatcctcaggtagtaagtattcattgtaaatctgatattatttgtattattatacctacctaatttgcagtgcagatcattgagataattaaaattataaccatctcgcaaataaataagtattttactgttttcgtaacagttttgtaataaaaaaacctataaatcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 7 - DNA, 2137 bp, unidentified
  source 1..2137: mol_type = other DNA; note = artificial sequence
gggagttcagtcgtcgaatgcaaaacgtaaaaaatattaataaggtaaaaactacagctggatcctcaggtagtaagtattcattgtaaatctgatattatttgtattattatacctacctaatttgcagtgcagatcattgagataattaaaattataaccatctcgcaaataaataagtattttactgttttcgtaacagttttgtaataaaaaaacctataaatcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 8 - DNA, 2188 bp, unidentified
  source 1..2188: mol_type = other DNA; note = artificial sequence
gacctttaattcaacccaacacaatatattatagttaaataagaattattatcaaatcatttgtatattaattaaaatactatactgtaaattacattttatttacaatcggatcctcaggtagtaagtattcattgtaaatctgatattatttgtattattatacctacctaatttgcagtgcagatcattgagataattaaaattataaccatctcgcaaataaataagtattttactgttttcgtaacagttttgtaataaaaaaacctataaatcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 9 - DNA, 2379 bp, unidentified
  source 1..2379: mol_type = other DNA; note = artificial sequence
atcattgagataattaaaattataaccatctcgcaaataaataagtattttactgttttcgtaacagttttgtaataaaaaaacctataaatattccggattattcataccgtcccaccatcgggcgcggatcccagcaggtaagtatcgataactttgttttctttcacatttacaactccgacatacaaatatgaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcgtaagccgccagtaagtattcattgtaaatctgatattatttgtattattatacctacctaatttgcagtgcagggtaattttattactgtttggtccataaacacttgtttaccatttccttttttacaagttttaatattttctgcatatataaaacattatttattttgcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 10 - DNA, 2560 bp, unidentified
  source 1..2560: mol_type = other DNA; note = artificial sequence
atcattgagataattaaaattataaccatctcgcaaataaataagtattttactgttttcgtaacagttttgtaataaaaaaacctataaatattccggattattcataccgtcccaccatcgggcgcggatcccagcaggtaagtatcgataactttgttttctttcacatttacaactccgacatacaaatcacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaacattgtaaacgtgccggagcaacaatctccggagactgcggccgtgtgcaaaaattaaaagctgttgaataaactggaatcgagctcttacaacaaatccaacattgaccagctggccgttattgtaaataagccgccagtaagtattcattgtaaatctgatattatttgtattattatacctacctaatttgcagtgcagggtaattttattactgtttggtccataaacacttgtttaccatttccttttttacaagttttaatattttctgcatatataaaacattatttattttgcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 11 - DNA, 2294 bp, unidentified
  source 1..2294: mol_type = other DNA; note = artificial sequence
atcattgagataattaaaattataaccatctcgcaaataaataagtattttactgttttcgtaacagttttgtaataaaaaaacctataaatattccggattattcataccgtcccaccatcgggcgcggatcccagcaggtaagtatcgataactttgttttctttcacatttacaactccgacatacaaatatggggagttcagtcgtcgaatgcaaaacgtaaaaaatattaataaggtaaaaactacagcttaagccgccagtaagtattcattgtaaatctgatattatttgtattattatacctacctaatttgcagtgcagggtaattttattactgtttggtccataaacacttgtttaccatttccttttttacaagttttaatattttctgcatatataaaacattatttattttgcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 12 - DNA, 2345 bp, unidentified
  source 1..2345: mol_type = other DNA; note = artificial sequence
atcattgagataattaaaattataaccatctcgcaaataaataagtattttactgttttcgtaacagttttgtaataaaaaaacctataaatattccggattattcataccgtcccaccatcgggcgcggatcccagcaggtaagtatcgataactttgttttctttcacatttacaactccgacatacaaatatggacctttaattcaacccaacacaatatattatagttaaataagaattattatcaaatcatttgtatattaattaaaatactatactgtaaattacattttatttacaatctaagccgccagtaagtattcattgtaaatctgatattatttgtattattatacctacctaatttgcagtgcagggtaattttattactgtttggtccataaacacttgtttaccatttccttttttacaagttttaatattttctgcatatataaaacattatttattttgcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 13 - DNA, 2079 bp, unidentified
  source 1..2079: mol_type = other DNA; note = artificial sequence
atcattgagataattaaaattataaccatctcgcaaataaataagtattttactgttttcgtaacagttttgtaataaaaaaacctataaattggatcctcaggtagtaagtattcattgtaaatctgatattatttgtattattatacctacctaatttgcagtgcagcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 2130 bp, unidentified
  source 1..2130: mol_type = other DNA; note = artificial sequence
aataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcgggatcctcaggtagtaagtattcattgtaaatctgatattatttgtattattatacctacctaatttgcagtgcagcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 2314 bp, unidentified
  source 1..2314: mol_type = other DNA; note = artificial sequence
cacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaacattgtaaacgtgccggagcaacaatctccggagactgcggccgtgtgcaaaaattaaaagctgttgaataaactggaatcgagctcttacaacaaatccaacattgaccagctggccgttattgtaaaggatcctcaggtagtaagtattcattgtaaatctgatattatttgtattattatacctacctaatttgcagtgcagcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 16 - DNA, 2045 bp, unidentified
  source 1..2045: mol_type = other DNA; note = artificial sequence
gggagttcagtcgtcgaatgcaaaacgtaaaaaatattaataaggtaaaaactacagctggatcctcaggtagtaagtattcattgtaaatctgatattatttgtattattatacctacctaatttgcagtgcagcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 17 - DNA, 144 bp, unidentified
  source 1..144: mol_type = other DNA; note = artificial sequence
aataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcg

SEQ ID NO: 18 - DNA, 92 bp, unidentified
  source 1..92: mol_type = other DNA; note = artificial sequence
atcatggagataattaaaatgataaccatctcgcaaataaataagtattttactgttttcgtaacagttttgtaataaaaaaacctataaat

SEQ ID NO: 19 - DNA, 2222 bp, unidentified
  source 1..2222: mol_type = other DNA; note = artificial sequence
aataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcgggatcctcaggtagtaagtattcattgtaaatctgatattatttgtattattatacctacctaatttgcagtgcagatcatggagataattaaaatgataaccatctcgcaaataaataagtattttactgttttcgtaacagttttgtaataaaaaaacctataaatcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 20 - DNA, 328 bp, unidentified
  source 1..328: mol_type = other DNA; note = artificial sequence
cacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaacattgtaaacgtgccggagcaacaatctccggagactgcggccgtgtgcaaaaattaaaagctgttgaataaactggaatcgagctcttacaacaaatccaacattgaccagctggccgttattgtaaa

SEQ ID NO: 21 - DNA, 59 bp, unidentified
  source 1..59: mol_type = other DNA; note = artificial sequence
gggagttcagtcgtcgaatgcaaaacgtaaaaaatattaataaggtaaaaactacagct

SEQ ID NO: 22 - DNA, 110 bp, unidentified
  source 1..110: mol_type = other DNA; note = artificial sequence
gacctttaattcaacccaacacaatatattatagttaaataagaattattatcaaatcatttgtatattaattaaaatactatactgtaaattacattttatttacaatc

SEQ ID NO: 23 - DNA, 2114 bp, unidentified
  source 1..2114: mol_type = other DNA; note = artificial sequence
atcatggagataattaaaatgataaccatctcgcaaataaataagtattttactgttttcgtaacagttttgtaataaaaaaacctataaatattccggattattcataccgtcccaccatcgggcgcggatcctcaggtagtaagtattcattgtaaatctgatattatttgtattattatacctacctaatttgcagtgcagcccattacggcagtataaattcgttcattttggatattgtttcagtgccggggttttacgagattgtgattaaggtccccagcgaccttgacgggcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

(19) United States
(12) Patent Application Publication
(10) Pub. No.: US 2025/0043307 A1
(43) Pub. Date: Feb. 6, 2025

(54) EXPRESSION CASSETTE CONTAINING OVERLAPPING OPEN READING FRAMES AND APPLICATION

(71) Applicant: GENEVOYAGER (WUHAN) CO., LTD., Hubei (CN)

(72) Inventors: He XIAO, Hubei (CN); Xiaobin HE, Hubei (CN); Gang HUANG, Hubei (CN); Xing PAN, Hubei (CN); Yicheng ZHOU, Hubei (CN); Liang DU, Hubei (CN); Mengdie WANG, Hubei (CN); Huanhuan ZUO, Hubei (CN); Hao SUN, Hubei (CN)

(73) Assignee: GENEVOYAGER (WUHAN) CO., LTD., Hubei (CN)

(21) Appl. No.: 18/434,746

(22) Filed: Feb. 6, 2024

Related U.S. Application Data
(63) Continuation of application No. PCT/CN2023/141032, filed on Dec. 22, 2023.

(30) Foreign Application Priority Data
Aug. 3, 2023 (CN) 202310975187.6
Nov. 17, 2023 (CN) 202311544887.6

(51) Int. Cl. C12N 15/86; C12N 5/07

(52) U.S. Cl. CPC C12N 15/86; C12N 5/0601; C12N 2710/14022; C12N 2710/14043; C12N 2710/14052; C12N 2750/14144

(57) ABSTRACT

An expression cassette containing overlapping open reading frames and an application thereof are provided. The overlapping open reading frames are overlapping open reading frames of a first ORF and a second ORF and include in sequence from a 5′ end to a 3′ end: a first promoter at least used to drive gene transcription of the first ORF; a 5′ part of a gene of the first ORF; an intron; and a 3′ part of a gene of the second ORF, the intron including a second promoter used only to drive gene transcription of the second ORF. By arranging two promoters in a single expression cassette in the disclosure, the two promoters are used to drive the expression of proteins of the overlapping reading frames and regulate the relative expression time and expression intensity of different proteins.

### CROSS-REFERENCE TO RELATED APPLICATION

This application is a continuation of international application of PCT application serial no. PCT/CN2023/141032, filed on Dec. 22, 2023, which claims the priority benefit of China application no. 202310975187.6, filed on Aug. 3, 2023 and China application no. 202311544887.6, filed on Nov. 17, 2023. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jan. 29, 2024, is named 142172_SEQUENCELISTING and is 61,181 bytes in size.

### BACKGROUND OF THE INVENTION

### Technical Field

The disclosure belongs to the field of genetic engineering, and more specifically, relates to an expression cassette containing a gene with overlapping open reading frames and an application.

### Description of Related Art

Adeno-associated virus (AAV), also known as adeno-accompanied virus, belongs to the genus Dependentvirus in the family Parvoviridae, is the single-stranded DNA-deficient virus with the simplest structure discovered so far, and requires helper viruses (usually adenoviruses) to participate in replication. The AAV genome is a single-stranded linear DNA, approximately 4700 bp, and includes two upstream and downstream open reading frames (ORFs): Rep and Cap, located between 2 T-shaped inverted terminal repeats (ITR) composed of 145 nucleotides each, as shown in FIG. 1. The function of ITR is to serve as the origin of viral replication and packaging signal. The Rep gene is involved in viral replication and integration and encodes viral replication proteins. The Cap gene is responsible for encoding the three capsid proteins of the virus. Recombinant adeno-associated virus (rAVV) is one of the most promising vectors in the field of gene therapy due to its characteristics of wide host range, low immunogenicity, high safety, and capability of mediating long-term stable expression of exogenous genes in animals. With the approval of the first recombinant adeno-associated virus (rAAV)-mediated gene therapy, there is increasing demand for large-scale AAV vector manufacturing technology (Yla-Herttuala S., 2012, Mol Ther, 20:1831-1832).

At present, there are two main types of rAAV production systems: one is a conventional production system using mammalian cells (e.g., 293 cells, COS cells, HeLa cells, KB cells, etc.), and the other is a production system using insect cells. Herein, the production system using insect cells uses the baculovirus expression vectors (BEVs) carrying the rAAV vector genome, Rep protein, and Cap protein to infect Sf9 insect cells to produce rAAV virus. Taking advantage of the fact that Sf9 cells can express large amounts of recombinant proteins, this system uses insect baculovirus containing rAAV genome, AAV-Rep, and AAV-Cap genes to transduce Sf9 cells, allowing rAAV to be assembled in Sf9 cells, and the purpose of large-scale production of rAAV is ultimately achieved.

In this production system, promoters play an important role. The selected exogenous gene is placed under the control of a native promoter and then expressed during the infection cycle, releasing the protein during cell lysis. The promoter is recognized by RNA polymerase, and transcription begins. In ribonucleic acid (RNA) synthesis, the promoter can interact with the transcription factor that determines the start of transcription, controls the initiation time and extent of gene expression (transcription). It contains the core promoter region and the regulatory region, which is like a “switch” that determines the activity of the gene and thus controls which protein the cell starts to produce. The promoter itself has no compilation function, but it has the function of directing the translation of amino acids in the gene, just like a flag. The core part thereof is the RNA polymerase binding site upstream of the non-coding region. Currently, the most commonly used promoters in BEVS are the polyhedral pol H (polyhedrin) promoter and the p10 promoter, in addition to basic promoters and a few early promoters. The expression levels of the same exogenous gene under the control of different promoters are greatly different.

In order to achieve maximum expression levels, very late promoters are used most of the time to drive the expression of exogenous genes. Although this approach is usually successful when large amounts of recombinant protein are required, but in some cases, the use of alternative promoters with earlier or weaker expression may be beneficial to the quality and yield of the final protein. Proteins that aggregate during rapid expression and proteins that require advanced post-translational modifications have also been shown to yield ideal products through sustained and stable expression using early promoters.

Immediate early and early promoters are activated prior to viral DNA replication. The immediate early promoter (IE) is transcribed by host cell RNA polymerase II and also utilizes host transcription factors. The structural organization of the immediate early and early promoters is quite different from that of the late and very late baculovirus promoters, and is more similar to that of other eukaryotic promoters. The most striking feature of the baculovirus immediate early promoter is the conserved CAGT motif, which serves as the transcription start site and plays an important role in regulating downstream gene expression. In some cases, immediate early promoter contains a TATA-like motif and/or an initiation sequence. This region is recognized by host RNA polymerase II and allows transcription of immediate early viral genes immediately following infection. The IE1 promoter is an immediate early promoter and is the most commonly used constitutive promoter for baculovirus expression because it is active at all time stages as well as in uninfected insect cells. The 39 k promoter is an early promoter that is transactivated by the IE1 protein. When being co-expressed with the IE1 protein, the 39 k promoter can also be used transiently.

The late promoter is transcribed by a viral RNA polymerase that recognizes the conserved (A/G/T) TAAG late/very late promoter motif. The transcription initiates at the second nucleotide within this motif, and its presence is required for promoter activity. Studies investigating the function of the TAAG motif in late promoters have shown that mutations in this sequence disrupt promoter transcriptional activity. The very late promoter transcriptional activity peaks at 18 to 24 hours after infection and exceeds late promoter activity. At 24 hours after infection, the transcription level of the very late promoter exceeds that of all other promoters in cells.

The P10 protein is expressed at high levels very late in the infection cycle and is thought to play a role in inclusion maturation and cell lysis required for virus release. Weyer and Possee first define the 101 nucleotides upstream of the ATG of the p10 gene as the p10 promoter sequence and suggest that it be used for the expression of exogenous proteins. Since then, the p10 promoter has been widely used to produce proteins in BEVS, such as the production of cauliflower mosaic virus gene 1 and human interleukin 2. p10 can also be used to connect other promoters for co-expression.

In the AAV insect cell production system, for the Cap protein, Urabe et al. replace the start codon AUG of VP1 with the suboptimal start codon ACG to construct one single polycistronic mRNA. This polycistronic mRNA expresses all three AAV2 VP proteins without the need for splicing (Urabe et al., 2002, Hum Gene Ther, 13:1935-1943). However, there are many and increasing serotypes of AAV, and the modification method of Urabe et al. is not suitable for all serotypes. For instance, AAV5 particles produced in the baculovirus system of Urabe et al. using ACG as the start codon of the VP1 capsid protein have poor infectivity. In the method provided by Chinese patent CN106544325A, different suboptimal start codons CTG are used to enhance the expression of VP1. Although this design improves the infectivity of AAV5 particles, this method lacks flexibility in adjusting the relative content of VP1/VP2/VP3. Further, this method also appears to lack the flexibility necessary for serotype-specific sequence adjustment. In the method provided by Chinese patent CN101522903A, an artificial intron containing the polH promoter sequence is inserted into the open reading frame of VP1, and two promoters are used to express VP1 and VP2/VP3. Although this design can also achieve the expression of VP1/VP2/VP3 in the same reading frame, this method cannot effectively adjust the relative amounts of VP1/VP2/VP3. Moreover, the relative expression amounts of VP1/VP2/VP3 vary greatly among different serotypes, resulting in low production efficiency.

For the expression of the Rep protein, Urabe et al. construct two independent expression cassettes containing Rep78 and Rep52 and inserted them into the same baculovirus vector. Herein, Rep52 uses the polH promoter, and Rep78 uses the deltaIE1 promoter, which has weaker promoter activity than the polH promoter (Urabe et al., 2002, Hum Gene Ther, 13:1935-1943). However, the immediate early promoter deltaIE1 is active early in baculovirus infection, and early and abundant expression of the Rep78 protein in insect cells may have a negative impact on the yield of recombinant baculovirus (Urabe et al., 2006, Journal of Virology 80.4:1874-1885). Further, some studies have found that the AAV Rep protein expression method developed by Urabe et al. has the problem of unstable passaging of baculovirus vectors (Kohlbrenner et al., 2005, Mol. Ther.12:1217-25). In order to solve the problem of inherent passaging instability of two independent Rep expression cassettes, Chinese patent CN 103849629 A discloses a method for producing AAV in insect cells. The translation initiation codons of the AAV Rep78 protein is replaced with ACG, and the codon is partially skipped by scanning ribosomes so that the translation initiation can also occur at the start codon of the Rep52 protein further downstream. In this method, the expression of Rep78 and Rep52 proteins in the same reading frame can be achieved, and the stability of the rAAV vector produced in insect cells is significantly improved. However, this method lacks flexibility in adjusting the relative contents of Rep52/Rep78 proteins. The high stoichiometric ratio of Rep52/Rep78 proteins may be a key factor in the high yield of rAAV (Xiao, Xiao et al., 1998, Journal of Virology 72.3 (1998): 2224-2232). In a method for producing AAV in an insect cell disclosed in Chinese patent CN 113897396 A, the inventors achieve the expression of Rep78 and Rep52 proteins in the same reading frame by constructing a Rep expression cassette containing an intron regulatory sequence. In this way, the stability of the rAAV vector produced in the insect cell is improved, and further, the relative content of Rep52/Rep78 proteins can be flexibly adjusted by adjusting the intron splicing efficiency. However, there is a lack of flexibility in controlling the relative expression time and relative expression intensity of the two.

Therefore, the large-scale production of rAAV vectors using baculovirus in insect cells still needs to be further improved. Many studies have shown that earlier promoters may be more efficient in producing some exogenous proteins, particularly those that require extensive post-translational processing and/or secretion. This is because when the latest promoter is most active, the host cell's processing of the exogenous protein may be impaired. For complex viral particles, different proteins may be required in different amounts. By using different baculovirus promoters that are active at different stages of infection, control of the expression time and amount of the target protein can be achieved (Bruder et al., Viruses 2022, 14, 2670). However, the mechanism of synergy between different promoters is still unclear.

### SUMMARY OF THE INVENTION

In view of the above defects or improvement needs of the related art, the disclosure provides an expression cassette for a gene containing overlapping open reading frames and an application, aiming to better control the relative expression time and relative intensity of different proteins represented by Rep78 and Rep52 proteins by constructing an expression cassette containing different proteins located in the same overlapping open reading frame and by using different promoters to control the expression of different proteins, and the use of overlapping open reading frames to express proteins not only ensures the stability of a rAAV vector produced an in insect cell, but also improves the yield and quality of the produced rAAV.

To achieve the above, in an aspect of the disclosure, the disclosure provides an expression cassette of a gene containing overlapping open reading frames, including:
operably linked from a 5′ end to a 3′ end:
an artificially constructed sequence; and
an overlapping open reading frame lacking a first translation initiation codon,
where the artificially constructed sequence includes a first promoter and a second promoter independent of each other, and a complete sequence of the first translation initiation codon is not included between the first promoter and the second promoter.

Preferably, the gene is a parvovirus Rep gene, the first promoter is used to drive expression of a Rep78/68 protein and a Rep52/40 protein, and the second promoter is used to additionally drive the expression of the Rep52/40 protein.

Preferably, the gene is a parvovirus Cap gene, the first promoter is used to drive expression of a VP1 protein and a VP2/3 protein, and the second promoter is used to additionally drive the expression of the VP2/3 protein.

Preferably, the artificially constructed sequence further includes an intron, and the intron is used to form the first translation initiation codon in the artificially constructed sequence through alternative splicing during post-transcriptional processing.

Further preferably, the intron is located between any two nucleotides of ATG.

Further preferably, the first promoter precedes the 5′ end of the intron.

Further preferably, the second promoter is within the intron.

Preferably, the second promoter precedes the 5′ end of the first promoter.

Preferably, the first promoter is active before the second promoter.

Further preferably, the first promoter is deltalE1, IE1, 39K, or p6.9, and the second promoter is polH or p10.

Preferably, the first promoter is p10, and the second promoter is polH.

According to an aspect of the disclosure, the disclosure provides an optimized expression cassette for a gene containing overlapping open reading frames. The overlapping open reading frames are overlapping open reading frames of a first ORF and a second ORF and further include in sequence from a 5′ end to a 3′ end:
a first promoter used to drive gene transcription of the first ORF;
a 5′ part of a gene of the first ORF;
an intron; and
a 3′ part of a gene of the second ORF,
where the intron includes a second promoter used to drive gene transcription of the second ORF, the first promoter is an immediate early promoter, an early promoter, or a late promoter, and the second promoter is a very late promoter.

Preferably, the intron is located between any two nucleotides of the translation initiation codons ATG of the first ORF. The intron is used to form the first translation initiation codon in the artificially constructed sequence through alternative splicing during post-transcriptional processing.

Preferably, the intron is located between the translation initiation codons ATG of the first ORF and an exon of the first ORF.

Preferably, the first promoter is deltaIE1, IE1, 39K, or p6.9, and the second promoter is polH or p10.

Preferably, the expression cassette is used to encode a parvovirus Rep gene, the first ORF is used to encode a Rep68/Rep78 protein, and the second ORF is used to encode a Rep40/Rep52 protein.

Preferably, the expression cassette is used to encode a parvovirus Cap gene, the first ORF is used to encode a VP1 protein, and the second ORF is used to encode a P2/VP3 protein.

According to another aspect of the disclosure, the disclosure further provides a nucleic acid molecule including a first expression cassette, and the first expression cassette is any expression cassette of the above.

Preferably, a second expression cassette is further included, and the first expression cassette and the second expression cassette are respectively used to encode the parvovirus Cap gene and the parvovirus Rep gene.

Preferably, the nucleic acid molecule further includes an exogenous gene.

Further preferably, AAV inverted terminal repeats are provided at both ends of the exogenous gene.

Further preferably, the exogenous gene is a reporter gene, and the reporter gene is at least one of a chloramphenicol acetyltransferase encoding gene, a β-galactosidase encoding gene, a β-glucuronidase encoding gene, a Renilla luciferase encoding gene, an alkaline phosphatase encoding gene, a firefly luciferase encoding gene, a green fluorescent protein encoding gene, and a red fluorescent protein encoding gene.

Further preferably, the exogenous gene is a gene encoding a drug polypeptide, and the drug polypeptide is at least one of lipoprotein esterase, apolipoprotein, cytokine, interleukin, and interferon.

According to another aspect of the disclosure, the disclosure further provides a recombinant baculovirus vector including the expression cassette according to the above.

Preferably, the recombinant baculovirus vector is an insect cell compatible vector.

According to another aspect of the disclosure, the disclosure further provides an insect cell including the recombinant baculovirus vector according to the above.

Preferably, the insect cell is a Spodoptera frugiperda cell, a Trichopodia cell, a Drosophila cell, or a mosquito cell.

To sum up, in the technical solutions provided by the disclosure when being compared to the related art, two promoters are arranged in a single expression cassette to drive the expression of proteins of the overlapping reading frames and regulate the relative expression time and expression intensity of different proteins. The relative expression time and a wider range of relative expression intensity of the Rep78/68 protein and the Rep52/40 protein in the Rep gene, as well as the relative expression time and a wider range of relative expression intensity of the VP1 protein and the VP2/3 protein in the Cap gene can be better controlled in the disclosure. In this way, not only the efficiency of rAAV vector production in the insect cell is improved, but also the quality of the produced AAV is improved.

### BRIEF DESCRIPTION OF THE DRAWINGS

FIG. 1 is a schematic diagram of expression regulation of a Cap gene and a Rep gene in wild-type AAV.

FIG. 2 is a schematic diagram of expression regulation of a RepA-D gene expression cassette I in Examples 1 to 4.

FIG. 3 is a schematic diagram of expression regulation of a Cap gene expression cassette in Example 1.

FIG. 4 is a schematic diagram of expression regulation of a RepI-IV gene expression cassette II in Examples 5 to 8.

FIG. 5 is a schematic diagram of expression regulation of a Rep1-4 gene expression cassette III in Example 5.

FIG. 6 is a Western Blot detection diagram of a recombinant baculovirus vector AcRepA-D expressing a Rep protein in Examples 1 to 4.

FIG. 7 is a Western Blot detection diagram of a recombinant baculovirus vector AcRepI-IV expressing a Rep protein in Examples 5 to 8.

FIG. 8 is a Western Blot detection diagram of a recombinant baculovirus vector AcRep1-4 expressing a Rep protein in Comparative Examples 1 to 4.

FIG. 9 is a growth kinetic curve of sf9 cells infected with constructs CRI-0, CRI-1, and CRI-2 in Verification Example 1.

FIG. 10 is a schematic diagram of expression regulation of a RepE gene expression cassette in Example 9.

FIG. 11 is a schematic structural diagram of a RepF-H gene expression cassette in Examples 10 to 12.

FIG. 12 is a Western Blot detection diagram of a recombinant baculovirus vector AcRepE-H expressing a Rep protein in Examples 9 to 12.

FIG. 13 is a Western Blot detection diagram of a recombinant baculovirus vector AcRep5 expressing a Rep protein in Comparative Example 5.

### DETAILED DESCRIPTION OF THE EMBODIMENTS

In order to make the objectives, technical solutions, and advantages of the disclosure clearer and more comprehensible, the disclosure is further described in detail with reference to the drawings and embodiments. It should be understood that the specific embodiments described herein serve to explain the invention merely and are not used to limit the invention. In addition, the technical features involved in the various embodiments of the invention described below can be combined with each other as long as the technical features do not conflict with each other.

For ease of understanding, the terms appearing in this specific embodiment are defined as follows:

The term “polynucleotide”: refers to a biological macromolecule composed of single nucleotides. The polynucleotide molecule mentioned in the disclosure may be a deoxyribonucleic acid (DNA) molecule or a ribonucleic acid (RNA) molecule. A person having ordinary skill in the art knows that the RNA sequence is basically similar to the DNA sequence or has a certain degree of sequence identity. Thymine (T) in the DNA sequence can be considered equivalent to uracil (U) in the RNA sequence.

The term “operably linked”: refers to the connection of polynucleotide (or polypeptide) sequences in a functional relationship. Two nucleotide sequences are “operably linked” when the two nucleotide sequences are placed in a functional relationship. For instance, a transcriptional control sequence (e.g., a promoter) is operably linked to a gene's coding sequence if it affects the transcription of the gene's coding sequence.

The term “expression cassette”: refers to a nucleic acid construct including coding and regulatory sequences operably linked when introduced into a host cell, resulting in the transcription and/or translation of RNA or polypeptide, respectively. An expression cassette is understood to include a promoter allowing the initiation of transcription, the open reading frame of the gene of interest, and a transcription terminator. Generally, the promoter sequence is placed upstream of the gene of interest at a distance compatible with expression control. The “expression cassette” may be a multi-piece nucleic acid construct or a one-piece nucleic acid construct.

The term “open reading frame” (ORF): refers to the normal nucleotide sequence of a structural gene with the potential to encode a protein or polypeptide and begins with a start codon and ends with a stop codon with no stop codon in between to interrupt translation. On an mRNA chain, a ribosome starts translation from the start codon, synthesizes a polypeptide chain along the mRNA sequence, and continues to extend. When a stop codon is encountered, the extension reaction of the polypeptide chain is terminated.

The term “vector”: refers to a nucleic acid molecule designed to transport, transfer, and/or store genetic material, as well as to express genetic material and/or integrate genetic material into the chromosomal DNA of a host cell, such as a plasmid vector, a cosmid vector, an artificial chromosome, a phage vector, and other viral vectors. A vector usually consists of at least three basic units, namely a replication source, a selectable marker, and a multiple cloning site.

The term “intron”: also known as spacer sequence, refers to a non-coding segment in a gene or mRNA molecule and is an intervening sequence in the DNA of eukaryotic cells. The intronic sequence is transcribed in the mRNA precursor, is removed by splicing, and is ultimately no longer present in the mature mRNA molecule. Depending on whether the splicing process is spontaneous or processed by the spliceosome, introns are divided into self-splicing introns and spliceosomal introns. A self-splicing intron is a special type of intron, a ribozyme that can be cut out by its own action to leave the mRNA. The intron involved in the disclosure is a spliceosomal intron, and the splicing of such an intron requires the help of the spliceosome. There are a splicing donor sequence and a splicing acceptor sequence at both ends of the intron sequence, which are the sequences on both sides of the cutoff and rejoining sites. The spliceosome is a ribonucleoprotein complex dynamically composed of small nuclear RNAs (snRNAs) and protein factors. The spliceosome recognizes the splicing site of the mRNA precursor, catalyzes the splicing reaction, completely cuts out the intron, and then rejoins the upstream and downstream RNA sequences.

The intron mentioned in the disclosure can be a natural intron, or an intron that has been artificially modified and has splicing activity in insect cells, and the source thereof is not limited to insect cells.

The term “AAV serotype”: since the discovery of adeno-associated viruses (AAVs), more than 100 AAV serotypes or mutants have been isolated from adenovirus, humans, or primates, and some mammals, among which AAV1-9 is mainly used. The main difference between rAAV vectors of different serotypes is the capsid protein. Different AAV serotypes have certain differences in infection efficiency and tissue specificity.

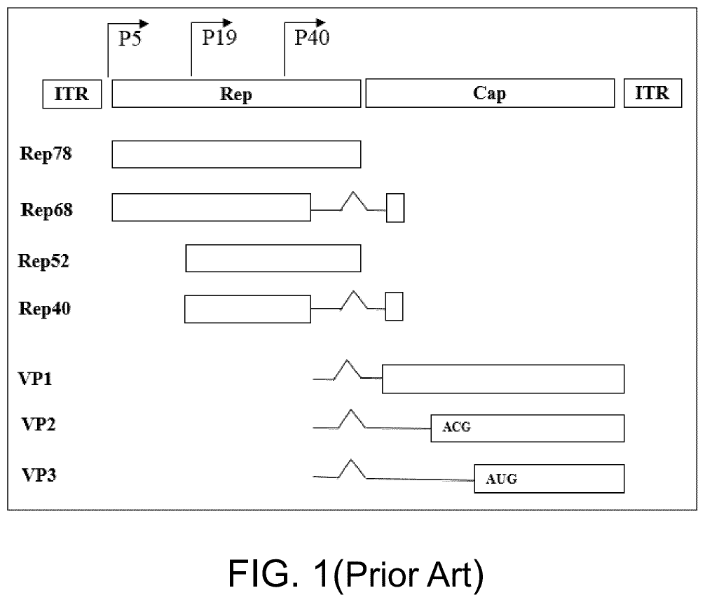

The genome structures of all known adeno-associated virus serotypes are very similar. AAV is a single-stranded DNA virus with a simple genome structure and a full length of approximately 4.7 kb, as shown in FIG. 1. Its genome contains a rep gene expression cassette, a cap gene expression cassette, and AAV inverted terminal repeats (ITRs) located at both ends of the genome. ITR is a 125-nucleotide palindrome structure at both ends of the genome, can form a self-complementary inverted T-shaped hairpin structure, and is a cis-acting element required for the initiation of DNA replication and packaging of recombinant AAV genome into infectious viral particles. Between the ITR sequences is the virus coding region, where the rep gene expression cassette and the cap gene expression cassette are located.

As a defective virus, AAV cannot replicate independently in the absence of helper viruses. Therefore, AAV can only be integrated into the host cell chromosome at a specific site and remain in a latent state. In the presence of helper virus, the increased expression amount of the rep gene can rescue the AAV genome integrated in the host cell chromosome and replicate AAV DNA in large quantities. The single-stranded rAAV genome is packaged into infectious virus particles under the action of VP capsid protein.

The term “Cap gene”: consists of overlapping amino acid sequences encoding the structural VP capsid protein and contains three overlapping open reading frames, encoding three types of subunits: VP1, VP2, and VP3. VP1, VP2, and VP3 contain different start codons and share a stop codon, and VP1 and VP2 share the VP3 sequence. The N-terminus of VP1 has a conserved phospholipase A2 sequence, and this sequence is involved in the escape of the virus from the body and is critical to its infectivity. The VP2 protein is not essential for assembly or infection. The core of the VP3 protein consists of a conserved β-barrel motif, which determines the differences in receptors between different serotypes of AAV and host cells. The correct ratio of the three proteins in wild-type AAV is 6:6:54, which is approximately 1:1:10.

The term “Rep gene”: is used to encode four overlapping multifunctional proteins, Rep78, Rep68, Rep52, and Rep40. Rep78 and Rep68 proteins are involved in the replication and integration of AAV and can bind to specific sequences in the ITR. Rep52 and Rep40 proteins have helicase and ATPase activities and participate in single-stranded genome replication and virus assembly. Whether in mammalian cells or insect cells, unspliced mRNA encoding Rep78 and Rep52 proteins is sufficient to prepare rAAV.

The term “relative expression content”: is the relative proportion of the expression amounts of different proteins. For instance, an increase in the relative expression content of Rep78/Rep52 protein may mean an increase in Rep78 protein expression amount, or it may mean a decrease in Rep52 protein expression amount.

The term “promoter”: is the DNA sequence located upstream of the 5′ end of a structural gene and is able to activate RNA polymerase, so that it can accurately bind to the template DNA and have the specificity for transcription initiation.

The term “solid rate”: taking the AAV vector as an example, the solid AAV is the AAV vector carrying the target gene. The empty shell AAV vector is an AAV vector that does not carry the target gene, consists of a serotype-specific AAV protein coat, and is usually produced by incomplete viral packaging during the production process. It must be minimized during purification of gene therapy products because these viral capsids do not contain DNA. When they are infused into patients, they compete for the limited number of receptors on the cell surface on the one hand, and they are not beneficial to gene therapy and may cause side effects on the other hand. Further, the increase in empty shell AAV content will lead to an increase in the dosage. The higher the dosage, the higher the risk of serious side effects. The same is true for AAVs that contain incomplete copies of therapeutic genes.

The solid rate refers to the proportion of solid AAV in the prepared AAV vector. The higher the solid rate, the greater the proportion of solid AAV, which proves that the quality of the prepared AAV vector is higher.

The disclosure provides an expression cassette used to express a gene including overlapping open reading frames in a cell, especially in an insect cell, and the expression cassette includes:
operably linked from a 5′ end to a 3′ end:
an artificially constructed sequence; and
an overlapping open reading frame lacking a first translation initiation codon,
where the artificially constructed sequence includes a first promoter and a second promoter independent of each other, and a complete sequence of the first translation initiation codon is not included between the first promoter and the second promoter.

In some embodiments, the artificially constructed sequence also includes an intron, and the intron is located between translation initiation codons ATG, for example, between AT and G or between A and TG. The intron is used to form the first translation initiation codon in the artificially constructed sequence through alternative splicing during post-transcriptional processing, so that the overlapping open reading frame can be expressed smoothly.

In some embodiments, the second promoter precedes the first promoter (i.e., precedes the 5′ end of the first promoter), and the first promoter precedes the intron. In some other embodiments, the first promoter precedes the intron, and the second promoter is contained within the intron.

In some embodiments, the first promoter is used to express all proteins encoded by the overlapping open reading frame, while the second promoter is used to adjust the relative expression time and relative expression content/expression intensity of these proteins. In some other embodiments, the gene encoded by the expression cassette is a parvovirus Rep gene. The two promoters of the disclosure may be used to adjust the relative expression time and relative expression content/expression intensity of different Rep proteins. Further, a Rep40 protein has a similar function to a Rep52 protein, and a Rep68 protein also has a similar function to a Rep78 protein. Therefore, the first promoter may be used to drive the expression of the Rep68 and/or Rep78 protein(s) and may also be used to drive the expression of the Rep40 protein and/or the Rep52 protein, and the second promoter is used to additionally drive the expression of the Rep40 protein and/or the Rep52 protein.

In some embodiments, the gene encoded by the expression cassette is a parvovirus Cap gene. The two promoters of the disclosure may be used to adjust the relative expression time and relative expression content/expression intensity of different Cap proteins. Further, the first promoter is used to drive the expression of a VP1 protein and may also be used to drive the expression of a VP2/3 protein, and the second promoter is used to additionally drive the expression of the VP2/3 protein.

In some embodiments, the first promoter is active before the second promoter. For instance, the first promoter may be an early and late promoter such as deltaIE1, IE1, 39K, or p6.9, and the second promoter may be a very late promoter such as p10 or polH.

In some other embodiments, the first promoter and the second promoter may produce activity almost simultaneously, for example, the first promoter is p10 and the second promoter is polH.

Since variations in some non-critical sites of the promoters have no impact on their functions, the sequences of the first promoter and the second promoter may also be partially different from the original sequences of the above promoters.

The disclosure further provides an optimized expression cassette for a gene including overlapping open reading frames for use in a cell, especially in an insect cell. The overlapping open reading frames are overlapping open reading frames of a first ORF and a second ORF and include in sequence from a 5′ end to a 3′ end:
a first promoter used to drive gene transcription of the first ORF;
a 5′ part of a gene of the first ORF;
an intron; and
a 3′ part of a gene of the second ORF,
where the intron includes a second promoter used to drive gene transcription of the second ORF, the first promoter is an immediate early promoter, an early promoter, or a late promoter, the second promoter is a very late promoter, and for example, in some embodiments, the first promoter is deltaIE1, IE1, 39K, or p6.9, and the second promoter is polH or p10.

In some embodiments, the intron is located between any two nucleotides of translation initiation codons ATG of the first ORF. The intron is used to form the first translation initiation codon in the artificially constructed sequence through alternative splicing during post-transcriptional processing.

In some other embodiments, the intron is located between the translation initiation codons ATG of the first ORF and an exon of the first ORF.

In some embodiments, the gene encoded by the expression cassette is a parvovirus Rep gene, the first ORF is used to encode the Rep78 protein, and the second ORF is used to encode the Rep52 protein. The two promoters of the disclosure may be used to adjust the relative expression time and relative expression content/expression intensity of different Rep proteins. Further, the Rep40 protein has a similar function to the Rep52 protein, and the Rep68 protein also has a similar function to the Rep78 protein. Therefore, the first promoter may be used to drive the expression of the Rep68 and/or Rep78 protein(s), and the second promoter is used to drive the expression of the Rep40 protein and/or the Rep52 protein.

In some embodiments, the second promoter contains translation initiation codon and stop codon sequences that are recognized by a ribosome, so that when the intron is not spliced during the post-transcriptional processing of a Rep gene expression cassette driven by the first promoter, the Rep40 protein and/or the Rep52 protein will not be translated and produced. In some embodiments, the gene encoded by the expression cassette is a parvovirus Cap gene. The two promoters of the disclosure may be used to adjust the relative expression time and relative expression content/expression intensity of different Cap proteins. Further, the first promoter is used to drive the expression of the VP1 protein, and the second promoter is used to additionally drive the expression of the VP2 and/or VP3 protein(s).

Since variations in some non-critical sites of the promoters have no impact on their functions, the sequences of the first promoter and the second promoter may also be partially different from the original sequences of the above promoters.

The expression cassette of the Rep gene may be integrated into different baculovirus vectors with the expression cassette of the Cap gene and a sequence of an exogenous gene containing AAV inverted terminal repeats at both ends, or they can be integrated into the same recombinant baculovirus vector. In some embodiments, both the expression cassette of the Rep gene and the expression cassette of the Cap gene can use the abovementioned expression cassette, or one of them can use the abovementioned expression cassette.

In some embodiments, the exogenous gene is a reporter gene and is, for example, a chloramphenicol acetyltransferase encoding gene, a β-galactosidase encoding gene, a β-glucuronidase encoding gene, a Renilla luciferase encoding gene, an alkaline phosphatase encoding gene, a firefly luciferase encoding gene, a green fluorescent protein encoding gene, or a red fluorescent protein encoding gene.

In some other embodiments, the exogenous gene is a gene encoding a drug polypeptide, and the drug polypeptide is lipoprotein esterase, apolipoprotein, cytokine, interleukin, or interferon and the like.

In some embodiments, the insect cell is a Spodoptera frugiperda cell, a Trichopodia cell, a Drosophila cell, or a mosquito cell and the like.

### Example 1

### Step One: Construction of Rep Gene Expression Cassette I and Recombinant Baculovirus Vector Containing Rep Gene Expression Cassette I

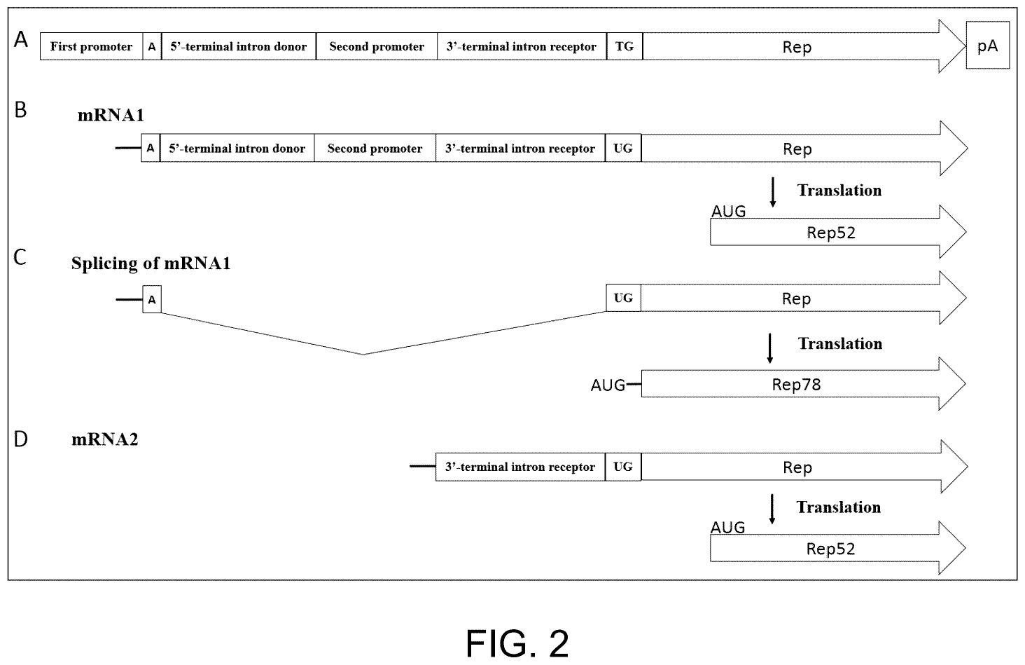

With reference to FIG. 2, herein, content A is a schematic diagram of a DNA chain of the Rep gene expression cassette I, content B is a schematic diagram of the translation and expression of the Rep52 protein when the intron is not spliced during post-transcriptional processing driven by the first promoter, content C is a schematic diagram of the translation and expression of the Rep78 protein after the intron is spliced during post-transcriptional processing driven by the first promoter, and content D is a schematic diagram of the translation and expression of the Rep78 protein after transcription driven by the second promoter. The gene expression cassette, from the 5′ end to the 3′ end, includes in sequence a first promoter sequence, adenine nucleotide (A), a 5′ end donor sequence of the intron, a second promoter sequence, a 3′ end acceptor sequence of the intron, thymine nucleotides, guanine nucleotides (TG), and a nucleotide sequence encoding the AAV serotype 2 Rep protein lacking only the translation initiation codons ATG of the Rep78 protein. The overlapping open reading frame nucleotide sequence used to encode the Rep protein is shown in SEQ ID No. 1, its entirety is used to encode the Rep78 protein, and the part used to encode the Rep52 protein is shown in SEQ ID No. 2. There are multiple nucleotide mutations between a Rep78 translation initiation codon and a Rep52 translation initiation codon to eliminate possible translation initiation sites in this region. Herein, deltaIE1 is used as the first promoter sequence and polH is used as the second promoter sequence in this example. The above sequences are connected through artificial direct synthesis or overlap extension PCR amplification to obtain a construct RepA, whose nucleotide sequence is shown in SEQ ID No. 3.

The construct is cloned into a pFastBac vector to prepare a transfer plasmid. The transfer plasmid is transformed into a DH10Bac strain, and a recombinant baculovirus vector AcRepA containing RepA is obtained through the gene transposition reaction mediated by Tn7 transposase.

### Step Two: Construction of Cap Gene Expression Cassette

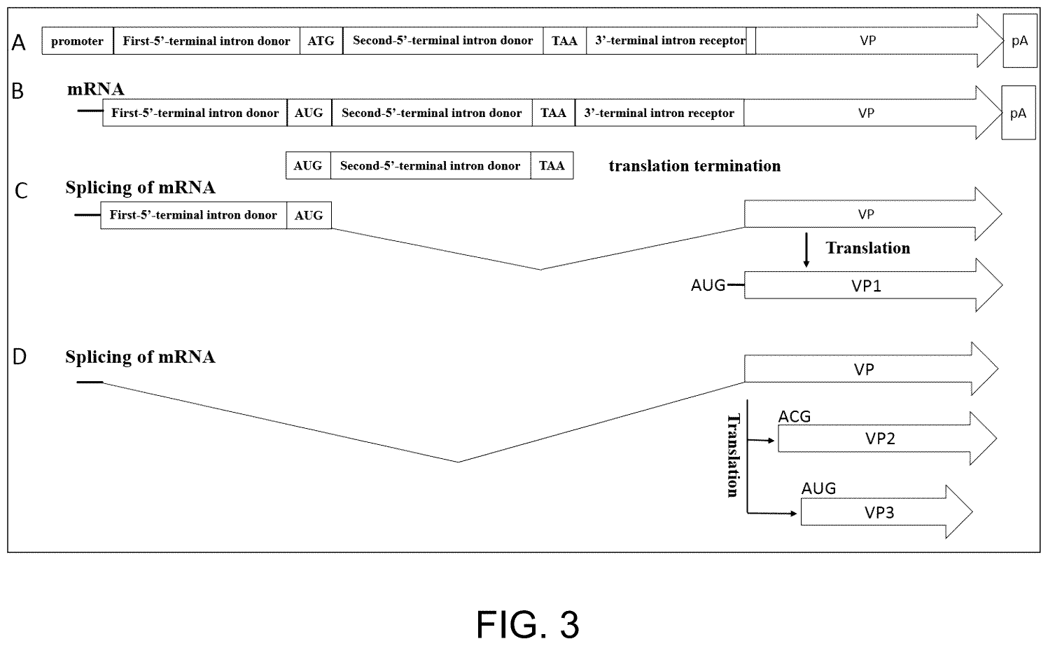

Construction of a Cap8 expression cassette containing the AAV serotype 8 Cap gene: The Cap gene expression cassette is constructed with reference to the method described in Example 3 of Chinese patent (CN202111105263.5). With reference to FIG. 3, the Cap8 expression cassette, from 5′ to 3′, includes in sequence, a p10 promoter, an artificially constructed sequence, and a nucleotide sequence encoding the AAV serotype 8 VP protein lacking only the translation initiation codons ATG of the VP1 protein. Herein, the artificially constructed sequence contains the translation initiation codons ATG and contains two intron alternative splicing sites. The amino terminus of the AAV serotype 8 VP protein is encoded with at least one nucleotide mutation to eliminate possible splicing sites within the coding sequence. The above sequences are connected through artificial direct synthesis or overlap extension PCR amplification to obtain a construct Cap8 expression cassette, whose nucleotide sequence is shown in SEQ ID No.4.

### Step Three: Construction of Recombinant AAV Bacmid

The method for constructing recombinant AAV bacmid for producing AAV virus in an insect cell is provided with reference to Example 1 in the previously applied patent CN112553257A in this example, and the following steps are included.

(1) A homologous recombination vector containing the essential functional elements Cap and Rep gene expression cassettes of AAV is constructed. The essential functional elements in the homologous recombination vector are then inserted into the C-terminus of the essential gene Ac135 (116492 . . . 117391) in the baculovirus genome in E. coli through Red homologous recombination. A recombinant baculovirus vector containing Cap and Rep gene expression cassettes is obtained, numbered Bac-Cap-Rep.

(2) A homologous recombination vector containing an ITR core element (ITR-GOI) is constructed. The ITR core element is numbered I-G-1, and its nucleotide sequence is shown in SEQ ID No. 5. The GOI in the ITR core element uses a red fluorescent protein mcherry gene expression cassette, that is, the miniEfla promoter controls mcherry expression to facilitate detection of rAAV activity in this example.

(3) The shuttle vector constructed in the above step (2) is used to transform competent cells containing the Bac-Cap-Rep recombinant bacmid. The essential functional elements in the homologous recombination vector are then inserted into the C-terminus of the essential gene Ac98 (88502 . . . 88464) in the baculovirus genome in E. coli through Red homologous recombination. A recombinant bacmid containing the functional protein components necessary for rAAV production and the ITR core element of the recombinant baculovirus genome is finally obtained, numbered Bac-Cap-Rep-ITR-GOI.

### Step Four: Preparation of AAV Recombinant Baculovirus

The recombinant AAV bacmid prepared in step three is transfected into a host cell line and cultured to obtain the AAV recombinant baculovirus, and the specific steps are provided as follows.

The above recombinant bacmid DNA was extracted and transfected into Sf9 insect cells to prepare recombinant baculovirus BEV and rAAV. The transfected Sf9 insect cells successfully produced BEV, and further infection with a large number of replicating and proliferating BEV caused obvious cytopathic effect (CPE) in Sf9 cells. The culture supernatant of Sf9 cells that had undergone CPE was collected, which contained a large amount of BEV, which was the 0^{th} generation BEV (P0). At the same time, the Sf9 cells containing a large amount of rAAV were collected. The prepared BEV-P0 was infected into suspension-cultured Sf9 cells at a multiplicity of infection (MOI=1). After 72 hours of infection, the cell activity dropped to less than 50%. The cell culture medium was centrifuged at 1000 g for 5 minutes, and the culture supernatant and cell pellet were collected. The supernatant was labeled as the first generation BEV-P1, and the cells were labeled as rAAV packaged with BEV-P0.

### Examples 2 to 4

In Examples 2 to 4, deltaIE1 in Example 1 is replaced with 39K, p6.9, and p10 as the first promoter sequence, and other steps are the same as in Example 1. Constructs RepB, RepC, and RepD, recombinant baculovirus vectors AcRepB, AcRepC, and AcRepD, as well as subsequent recombinant AAV bacmid and AAV recombinant baculovirus are obtained. The nucleotide sequences of RepB-RepD are shown in SEQ ID No. 6-8.

### Example 5

### Step One: Construction of Rep Gene Expression Cassette II and Recombinant Baculovirus Vector Containing Rep Gene Expression Cassette II

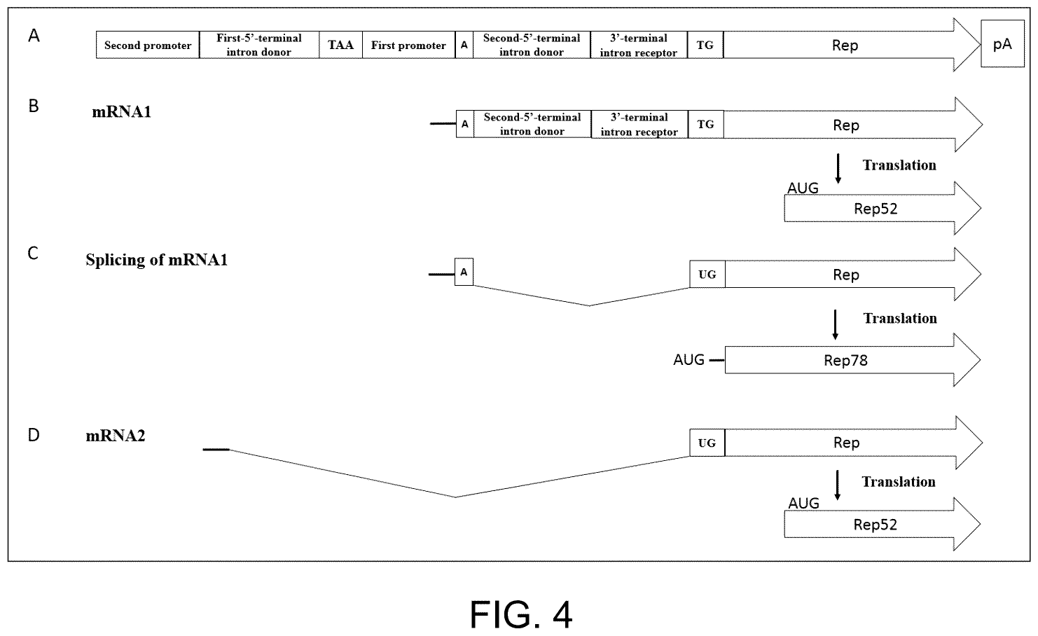

With reference to what is shown in FIG. 4, herein, content A is a schematic diagram of a DNA chain of the Rep gene expression cassette II, content B is a schematic diagram of the translation and expression of the Rep52 protein when the intron is not spliced during post-transcriptional processing driven by the first promoter, content C is a schematic diagram of the translation and expression of the Rep78 protein after the intron is spliced during post-transcriptional processing driven by the first promoter, and content D is a schematic diagram of the translation and expression of the Rep52 protein after splicing of the first intron splicing donor and splicing acceptor during post-transcriptional processing driven by the second promoter. A TAA translation termination sequence is inserted between the first promoter and the second promoter. During post-transcriptional processing driven by the second promoter, if the intron is not spliced or the second intron splicing donor and splicing acceptor are spliced, protein translation will be terminated prematurely and the Rep78 protein will not be produced. The gene expression cassette, from the 5′ end to the 3′ end, includes in sequence a second promoter sequence, a 5′ end donor sequence of the first intron, a first promoter sequence, adenine nucleotide (A), a 5′ end donor sequence of the second intron, a 3′ end acceptor sequence of the intron, thymine nucleotides, guanine nucleotides (TG), and a nucleotide sequence encoding the AAV serotype 2 Rep protein lacking only the translation initiation codons ATG of the Rep78 protein. Herein, deltaIE1 is used as the first promoter sequence and polH is used as the second promoter sequence in this example. The above sequences are connected through artificial direct synthesis or overlap extension PCR amplification to obtain a construct RepI, whose nucleotide sequence is shown in SEQ ID No. 9.

The construct was cloned into a pFastBac vector to prepare a transfer plasmid. The transfer plasmid was transformed into a DH10Bac strain, and a recombinant baculovirus vector AcRepI containing RepI was obtained through the gene transposition reaction mediated by Tn7 transposase.

Step two to step four are the same as in Example 1.

### Examples 6 to 8

In Examples 6 to 8, deltaIE1 in Example 5 is replaced with 39K, p6.9, and p10 as the first promoter sequence, and other steps are the same as in Example 5. Constructs RepII, RepIII, and RepIV (the nucleotide sequences of which are shown in SEQ ID No. 10-12), recombinant baculovirus vectors AcRepII, AcRepIII, and AcRepIV as well as subsequent recombinant AAV bacmid and AAV recombinant baculovirus are obtained.

### Comparative Examples 1 to 4

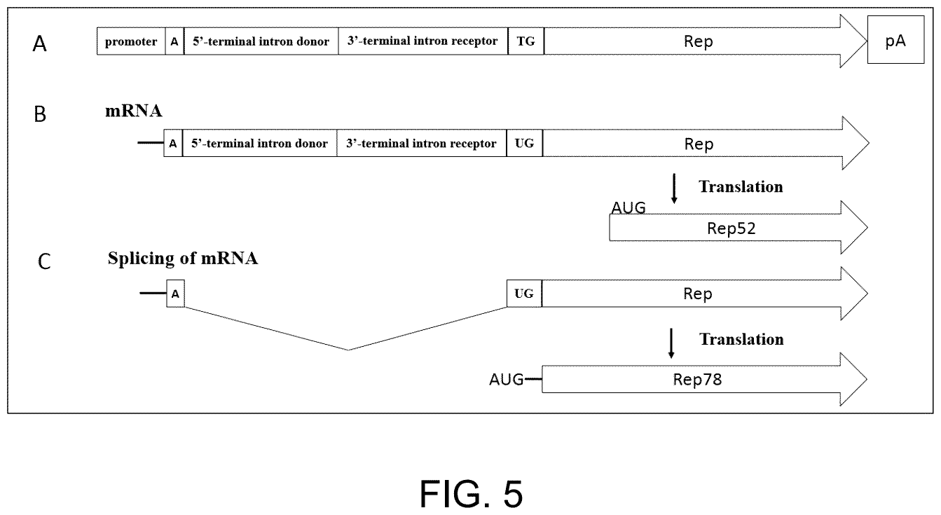

Step one: A Rep gene expression cassette III and a recombinant baculovirus vector containing the Rep gene expression cassette III are constructed, and the Rep gene expression cassette III is constructed with reference to the method described in Example 6 of Chinese patent (CN202111105263.5). With reference to FIG. 5, herein, content A is a schematic diagram of a DNA chain of the Rep gene expression cassette III, content B is a schematic diagram of the translation and expression of the Rep52 protein when the intron is not spliced during post-transcriptional processing, and content C is a schematic diagram of the translation and expression of the Rep78 protein after the intron is spliced during post-transcriptional processing. The gene expression cassette, from the 5′ end to the 3′ end, includes in sequence a promoter sequence, adenine nucleotide (A), a 5′ end donor sequence of the intron, a 3′ end acceptor sequence of the intron, thymine nucleotides, guanine nucleotides (TG), and a nucleotide sequence encoding the AAV serotype 2 Rep protein lacking the translation initiation codons ATG of the Rep78 protein. Herein, Comparative Examples 1 to 4 use deltaIE1, 39K, p6.9, and polH as promoter sequences. The above sequences are connected through artificial direct synthesis or overlap extension PCR amplification to obtain the constructs Rep1-4, whose nucleotide sequences are shown in SEQ ID No. 13-16.

The constructs were cloned into a pFastBac vector to prepare a transfer plasmid. The transfer plasmid was transformed into a DH10Bac strain, and recombinant baculovirus vectors AcRep1-4 respectively containing Rep1-4 were obtained through the gene transposition reaction mediated by Tn7 transposase.

### Step Two to Step Four are the Same as in Example 1.

### Experimental Result Verification

For the ease of explanation of the experimental results, the recombinant AAV bacmids (Bac-Cap-Rep-ITR-GOI) constructed according to Examples 1 to 6 and Comparative Example 4 of the disclosure are numbered according to Table 1 according to their corresponding Cap gene expression cassette, Rep gene expression cassette, and ITR core element. The 7 different recombinant AAV bacmids constructed in this example all contain the Cap gene expression cassette, the Rep gene expression cassette, and the ITR core element. Herein, the recombinant AAV bacmid CRI-0 is a control bacmid containing the Rep4 gene expression cassette, and the Rep78/52 protein in the Rep4 gene expression cassette is driven by a single very late strong promoter polH. The details are shown in Table 1.

TABLE 1
List of components of 7 different recombinant AAV bacmids
constructed in the Examples and Comparative Examples

| | Functional protein components | | |
|---|---|---|---|
| | and ITR core elements | | |
| | necessary for rAAV production | | |
| Recombinant AAV | Cap gene | Rep gene | ITR core |
| bacmid (Bac-Cap-Rep- | expression | expression | component |
| ITR-GOI) number | cassette | cassette | (ITR-GOI) |
| CRI-0 | Cap8 | Rep4 | I-G-1 |
| CRI-1 | Cap8 | RepA | I-G-1 |
| CRI-2 | Cap8 | RepB | I-G-1 |
| CRI-3 | Cap8 | RepC | I-G-1 |
| CRI-4 | Cap8 | RepD | I-G-1 |
| CRI-5 | Cap8 | RepI | I-G-1 |
| CRI-6 | Cap8 | RepII | I-G-1 |

Verification Example 1 detects the expression of the Rep proteins (Rep78 and Rep52), and the specific steps are as follows.

The above recombinant baculovirus vector DNA was extracted and transfected into Sf9 insect cells to prepare the recombinant baculovirus BEV. The transfected Sf9 insect cells successfully produced BEV, and further infection with a large number of replicating and proliferating BEV caused obvious cytopathic effect (CPE) in Sf9 cells. The culture supernatant of Sf9 cells that had undergone CPE was collected, which contained a large amount of BEV, which was the 0th generation BEV (P0). At the same time, the Sf9 cells containing a large amount of rAAV were collected. The prepared BEV-P0 was infected into suspension-cultured Sf9 cells at a multiplicity of infection (MOI=1). After 72 hours of infection, the cell activity dropped to less than 50%. The cell culture medium was centrifuged at 1000 g for 5 minutes, and the culture supernatant and cell pellet were collected. The supernatant was labeled as the first generation BEV-P1. Continuing to expand the culture, the prepared BEV-P1 was infected into suspension-cultured Sf9 cells at a multiplicity of infection (MOI=1). After 72 hours of infection, the cell activity dropped to less than 50%. The cell culture medium was centrifuged at 1000 g for 5 minutes, and the cell pellet was collected for Western Blot to detect the expression of the Rep proteins (Rep78 and Rep52).

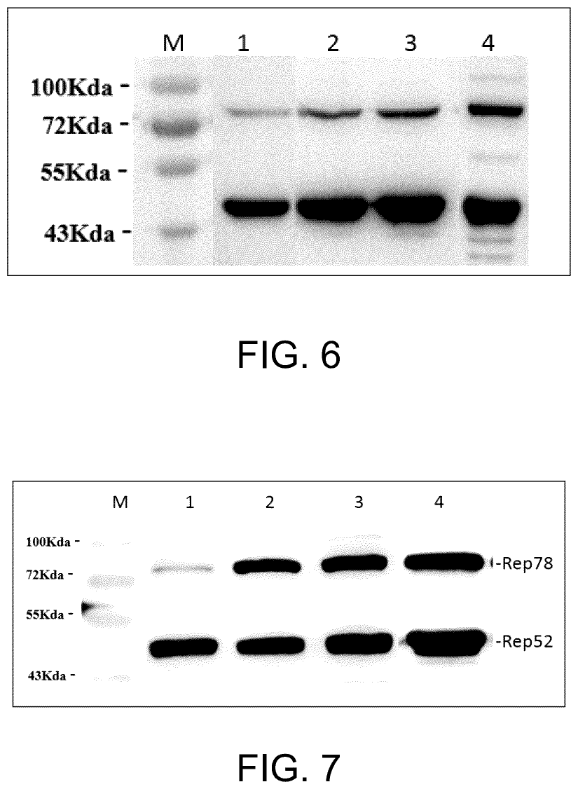

FIGS. 6 to 8 are Western Blot detection diagrams of the Rep proteins (Rep78 and Rep52) of the recombinant baculovirus vector containing the Rep gene expression cassette in Examples 1 to 8 and Comparative Examples 1 to 4.

FIG. 6 is a Western Blot detection diagram of the Rep proteins (Rep78 and Rep52) of the recombinant baculovirus vectors AcRepA-D containing the Rep gene expression cassette I, in which lanes 1˜4 represent AcRepA-D respectively. It can be seen from FIG. 6 that the above recombinant baculovirus vectors can produce the Rep78 and Rep52 proteins, and the expression amount of the Rep52 protein is significantly higher than that of the Rep78 protein. The high stoichiometric ratio of Rep52 to Rep78 may be a key factor for high rAAV yield. In constructs AcRepA, AcRepB, AcRepC, and AcRepD, the inventors insert the second promoter into the intron sequence to relatively enhance the expression of the Rep52 protein.

FIG. 7 is a Western Blot detection diagram of the Rep proteins (Rep78 and Rep52) of the recombinant baculovirus vectors AcRepI, AcRepII, AcRepIII, and AcRepIV containing the Rep gene expression cassette II, in which lanes 1-4 represent AcRepI-IV respectively. In constructs AcRepI, AcRepII, AcRepIII, and AcRepIV, the inserted second promoter drives the splicing interaction between the first intron splicing donor and the splicing acceptor during post-transcriptional processing to enhance the expression of the Rep52 protein.

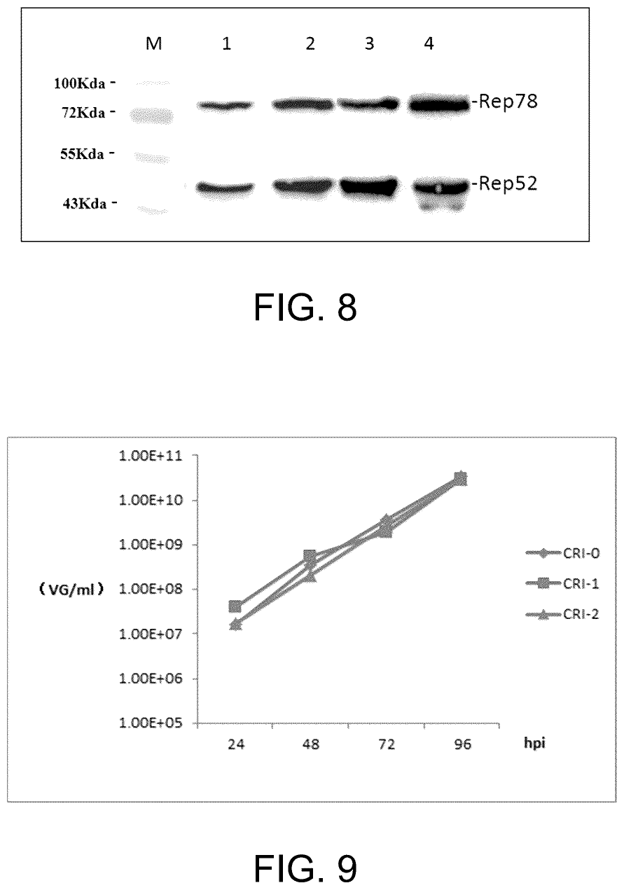

FIG. 8 shows the recombinant baculovirus vectors AcRep1-4 containing the Rep gene expression cassette III in Comparative Examples 1-4, in which lanes 1-4 respectively represent the AcRep1-4 in Comparative Examples. It can be seen from the figure that the above recombinant baculovirus vectors can all produce the Rep78 and Rep52 proteins. In constructs AcRep1, AcRep2, AcRep3, and AcRep4, the Rep78/52 protein is expressed through a single promoter. Herein, AcRep1, AcRep2, and AcRep3 use deltaIE1, 39K, and p6.9 promoters to drive the expression of Rep78/52 protein in the immediate early, delayed early, and late stages of baculovirus respectively. Early expression of the Rep78 protein may be beneficial for AAV genome replication and packaging. However, compared with the very late promoter (p10 or polH), the lower promoter strength of deltaIE1, 39K, and p6.9 results in insufficient Rep52 protein, which is not conducive to high rAAV productivity, so it is necessary to enhance the expression of the Rep52 protein.

### Verification Example 2 Growth Kinetics Analysis

The immediate early promoter deltaIE1 is active early in baculovirus infection, and early and abundant expression of the Rep78 protein in insect cells may have a negative impact on the yield of recombinant baculovirus (Urabe et al., 2006, Journal of Virology 80.4:1874-1885). In order to compare the growth kinetics of the recombinant baculoviruses containing different Rep gene expression cassettes in the above Examples and Comparative Examples, the above recombinant baculovirus is used to infect Sf9 cells at a multiplicity of infection (MOI=0.5). The supernatant of the infected cell culture is collected at 24, 48, 72, and 96 hours after infection, and the titer of the second generation BEV-P2 is determined using fluorescence quantitative PCR (qPCR), where the titer unit is expressed in VG/ml (VG, virus genomes). A pair of primers (Q-GP64-F: AACTTGGACATTACCCCGCC (SEQ ID NO: 24) and Q-GP64-R: CCGTTGTACGCATACGCCTG (SEQ ID NO: 25)) corresponding to the gp64 gene is used to determine the BEV titer, and titer unit is expressed in VG/ml (VG, virus genomes). The experimental results are shown in FIG. 9. In constructs CRI-0, CRI-1, and CRI-2, the Rep78 protein is expressed in the very late stage, immediate early stage, and delayed early stage respectively. As can be seen from FIG. 9, there are no significant differences in viral titers among CRI-0, CRI-1, and CRI-2, indicating that the early expression of the Rep78 protein in CRI-1 and CRI-2 constructs has less impact on baculovirus replication. In the Rep expression cassette designed by Urabe et al., deltaIE1 alone drives the expression of the Rep78 protein. In CRI-1, the rep gene driven by deltalE1 promoter is partially transcribed and translated into the Rep78 protein through intron splicing, and partially transcribed and translated into the Rep52 protein, resulting in a relatively lower expression amount of the Rep78 protein, which may be the reason for its reduced impact on baculovirus replication.

### Verification Example 3: Purification of Recombinant AAV Virus Particles and Detection of rAAV Virus Titer and Virus Particle Solid Rate

This verification example uses Q-PCR to detect the titer of the harvested rAAV virus, and the titer unit is expressed in VG/ml (VG, virus genomes). rAAV titers are measured using a pair of primers (Q-ITR-F: GGAACCCCTAGTGATGGAGTT (SEQ ID NO: 26) and Q-ITR-R: CGGCCTCAGTGAGCGA (SEQ ID NO: 27)) targeting the ITR sequence. The specific operation is as follows. The culture was continuously expanded according to step four of Example 1 until the Sf9 cells cultured in the suspension were infected with BEV-P2 seed virus according to the multiplicity of infection (MOI=1) for rAAV packaging. The packaging volume is 300 mL to 400 mL. After 3 days of infection, cell activity was monitored. After the activity was lower than 50%, the cell culture was harvested, 500 μl of the harvested mixed solution (mixed solution of cells and supernatant) was put into a 1.5 ml EP tube, frozen and thawed four times in liquid nitrogen, and treated in a 37° C. water bath. 0.1 μl of Benzonase was added to the 500 μl mixed solution, and the mixed solution was mixed in a 37° C. water bath for 1 hour and then in a 95° C. water bath for 10 minutes. Centrifugation was performed at 2500 g for 10 minutes, and the supernatant was collected in a new 1.5 ml EP tube. 200 μl of the supernatant was added to 10 μl of 10% SDS solution to a final concentration of 0.5% SDS. 1.2 μl Proteinase K (working concentration 112 μg/ml) was then added, the final volume was approximately 210 μl, and a 55° C. water bath was performed for 1 hour. After a short centrifugation, a 95° C. water bath was performed for 10 minutes. After mixing, 5 μl of sample was taken out and 145 μl of ddH₂O was added to make the final volume 150 μl. After mixing, 10 μl of the sample was taken out and 90 μl of ddH₂O was added to make the final volume 100 μl. 2 μl was used as a Q-PCR template to measure the titer of the virus sample. The experimental results are shown in Table 2.

This example uses analytical ultracentrifugation technology (abbreviated as AUC) to detect the solid rate (complete capsid: total capsid) of the recombinant adeno-associated virus (rAAV).

With the use of the sedimentation rate analysis method in the analytical ultracentrifugation technology, the sample is rotated at high speed in the centrifuge, and each component in the sample moves to the bottom of the sample pool. Different components have different movement rates (i.e., sedimentation coefficients) and have different times to fall to the bottom during centrifugation. During this process, the sample distribution state is scanned, and the sedimentation properties of different components are obtained by analyzing its change process over time. The sedimentation coefficient of each component of the sample depends on the molecular weight, molecular shape, and conformation. AUC is generally used to characterize the solid rate of rAAV.

The collected cell culture was purified using an affinity chromatography column (AVIPure AAV8 Resin). 400 μl of the purified rAAV sample was diluted to a dilution factor of OD₂₃₀=0.90, and loaded onto the sample. The sedimentation coefficient and peak area percentage of each peak were recorded. Herein, the sedimentation coefficient of empty shell virus was 55S to 65S, the sedimentation coefficient of solid virus was 80S to 110S, 65S to 80S was the partial part, and greater than 110S was the aggregate. The peaks with sedimentation coefficients between 20S and 150S were normalized to obtain the peak area percentage of each peak, and the empty and solid rate of the virus sample was reported based on the results. The experimental results are shown in Table 2.

TABLE 2
Titer and solid rate detection results of rAAV virions
produced using 7 different recombinant AAV bacmids

| Recombinant | rAAV | rAAV solid rate |
|---|---|---|
| AAV bacmid | virus titer | intact capsids: |
| number | (VG/ml) | total capsids) |
| CRI-0 | 1.12E+12 | 24% |
| CRI-1 | 1.23E+12 | 37% |
| CRI-2 | 1.37E+12 | 45% |
| CRI-3 | 7.37E+11 | 28% |
| CRI-4 | 6.41E+11 | 27% |
| CRI-5 | 1.20E+12 | 32% |
| CRI-6 | 1.27E+12 | 43% |

As can be seen from Table 2, the titers of rAAV virus particles produced using recombinant AAV bacmids CRI-1, CRI-2, CRI-5, and CRI-6 are higher than those of the control bacmid CRI-0, and the solid rate is significantly higher than that of the control bacmid CRI-0. Compared to the control bacmid CRI-0, the early expression of the Rep78 protein and the higher Rep52/Rep78 ratio in bacmids CRI-1, CRI-2, CRI-5, and CRI-6 may be the key factors for their high titers and the high solid rate. The titers of rAAV virus particles produced using the recombinant AAV bacmids CRI-3 and CRI-4 are lower than that of the control bacmid CRI-0, but their solid rates are higher than that of the control bacmid CRI-0. Bacmids CRI-0, CRI-3, and CRI-4 use polH, p6.9, and p10 late promoters respectively to drive the expression of the Rep78 protein, where the polH promoter is the most active. Compared to the control bacmid CRI-0, the lower Rep78 protein expression and higher Rep52/Rep78 ratio in bacmids CRI-3 and CRI-4 may be responsible for their relatively low titers and high solid rates. Increasing the solid rates of viral particles is beneficial to the rAAV product, so fewer particles can be used to obtain a similar amount of genome copies per kilogram, and it is also beneficial to establish a stable downstream purification process.

### Example 9

### Step One: Construction of Rep Gene Expression Cassette IV and Recombinant Baculovirus Vector Containing Rep Gene Expression Cassette IV

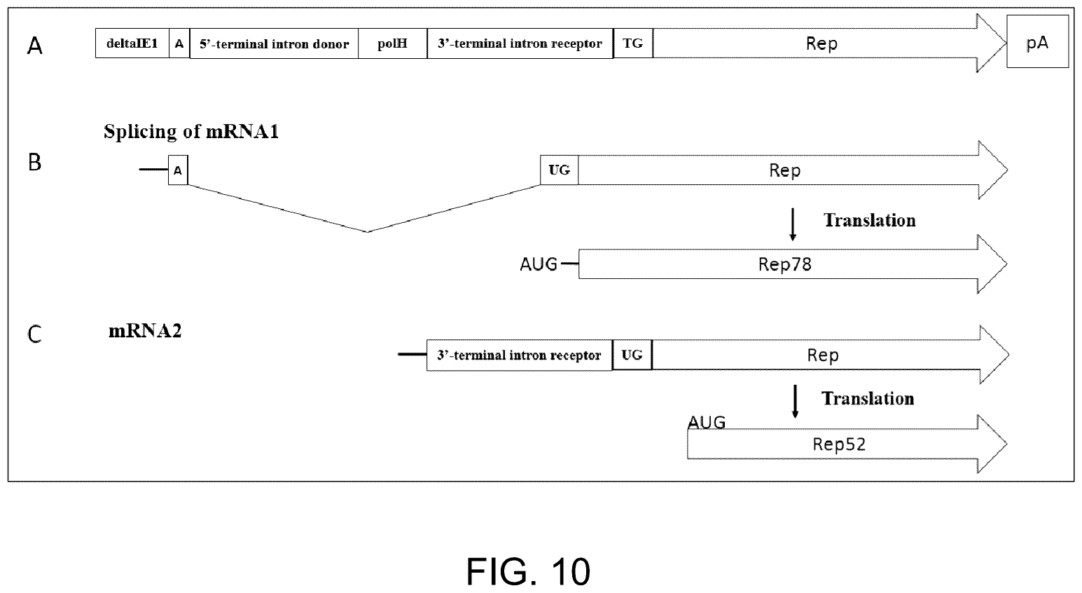

With reference to FIG. 10, herein, content A is a schematic diagram of a DNA chain of the Rep gene expression cassette IV, content B is a schematic diagram of the translation and expression of the Rep78 protein after the intron is spliced during post-transcriptional processing driven by the first promoter, and content C is a schematic diagram of the translation and expression of the Rep78 protein after transcription driven by the second promoter. The gene expression cassette, from the 5′ end to the 3′ end, includes in sequence a first promoter sequence, adenine nucleotide (A), a 5′ end donor sequence of the intron, a second promoter sequence, a 3′ end acceptor sequence of the intron, thymine nucleotides, guanine nucleotides (TG), and a nucleotide sequence encoding the AAV serotype 2 Rep protein lacking only the translation initiation codons ATG of the Rep78 protein. The overlapping open reading frame nucleotide sequence used to encode the Rep protein is shown in SEQ ID No. 1, its entirety is used to encode the Rep78 protein, and the part used to encode the Rep52 protein is shown in SEQ ID No. 2. There are multiple nucleotide mutations between a Rep78 translation initiation codon and a Rep52 translation initiation codon to eliminate possible translation initiation sites in this region. In this example, deltaIE1 is used as the first promoter sequence (the nucleotide sequence is shown in SEQ ID No. 17), and polH is used as the second promoter sequence (the nucleotide sequence is shown in SEQ ID No. 18). Herein, the second promoter contains the translation initiation and stop codon sequences that are recognized by a ribosome, so that when the intron is not spliced during the post-transcriptional processing of a Rep gene expression cassette driven by the first promoter, the Rep52 protein will not be translated and produced. The above sequences are connected through artificial direct synthesis or overlap extension PCR amplification to obtain a construct RepE, whose nucleotide sequence is shown in SEQ ID No. 19.

The construct was cloned into a pFastBac vector to prepare a transfer plasmid. The transfer plasmid was transformed into a DH10Bac strain, and a recombinant baculovirus vector AcRepE containing RepE was obtained through the gene transposition reaction mediated by Tn7 transposase.
Step two: Construction of Cap gene expression cassette
This step is the same as step two of Example 1.
Step three: Construction of recombinant AAV bacmid
This step is the same as step three of Example 1.
Step four: Preparation of AAV recombinant baculovirus
This step is the same as step four of Example 1.

### Examples 10 to 12

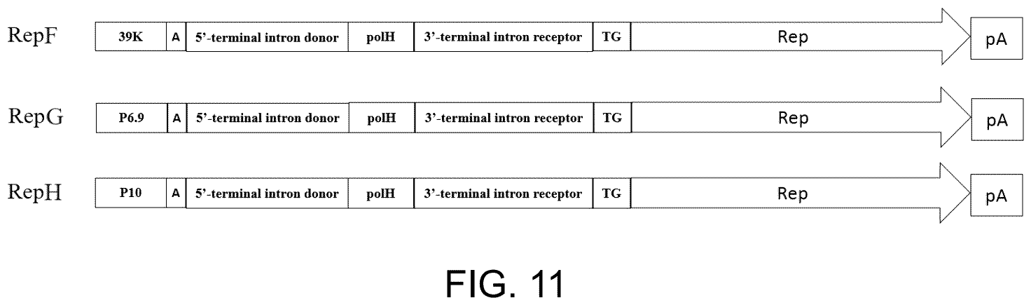

In Examples 10 to 12, deltaIE1 in Example 9 is replaced with 39K (SEQ ID No. 20), p6.9 (SEQ ID No. 21), and p10 (SEQ ID No. 22) as the first promoter sequence, and other steps are the same as in Example 9. Constructs RepF, RepG, and RepH, as shown in FIG. 11, corresponding recombinant baculovirus vectors AcRepF, AcRepG, and AcRepH, as well as subsequent recombinant AAV bacmid and AAV recombinant baculovirus are obtained.

### Comparative Example 5

Step one: A Rep gene expression cassette V and a recombinant baculovirus vector containing the Rep gene expression cassette V are constructed, and the Rep gene expression cassette V is constructed with reference to the method described in Example 6 of Chinese patent (CN202111105263.5). With reference to FIG. 5, herein, content A is a schematic diagram of a DNA chain of the Rep gene expression cassette V, content B is a schematic diagram of the translation and expression of the Rep52 protein when the intron is not spliced during post-transcriptional processing, and content C is a schematic diagram of the translation and expression of the Rep78 protein after the intron is spliced during post-transcriptional processing. The gene expression cassette, from the 5′ end to the 3′ end, includes in sequence a promoter sequence, adenine nucleotide (A), a 5′ end donor sequence of the intron, a 3′ end acceptor sequence of the intron, thymine nucleotides, guanine nucleotides (TG), and a nucleotide sequence encoding the AAV serotype 2 Rep protein lacking the translation initiation codons ATG of the Rep78 protein. Herein, Comparative Example 5 uses polH as the promoter sequence. The above sequences are connected through artificial direct synthesis or overlap extension PCR amplification to obtain a construct Rep5, whose nucleotide sequence is shown in SEQ ID No. 23.

The construct was cloned into a pFastBac vector to prepare a transfer plasmid. The transfer plasmid was transformed into a DH10Bac strain, and a recombinant baculovirus vector AcRep5 containing Rep5 was obtained through the gene transposition reaction mediated by Tn7 transposase.

### Step Two to Step Four are the Same as in Example 9.

### Experimental Result Verification

For the ease of explanation of the experimental results, the recombinant AAV bacmids (Bac-Cap-Rep-ITR-GOI) constructed according to Examples 9 to 12 and Comparative Example 5 of the disclosure are numbered according to Table 3 according to their corresponding Cap gene expression cassette, Rep gene expression cassette, and ITR core element. The 5 different recombinant AAV bacmids constructed in this example all contain the Cap gene expression cassette, the Rep gene expression cassette, and the ITR core element. Herein, the recombinant AAV bacmid CRI-7 is a control bacmid containing the Rep5 gene expression cassette, and the Rep78/52 protein in the Rep5 gene expression cassette is driven by a single very late strong promoter polH. The details are shown in Table 3.

TABLE 3
List of components of 7 different recombinant AAV
bacmids constructed in the Examples and Comparative Example 5

| | | Functional protein components | | |
|---|---|---|---|---|
| | Recombinant | and ITR core elements necessary | | |
| Corresponding | AAV bacmid | for rAAV production | | |
| Example or | (Bac-Cap-Rep- | Cap gene | Rep gene | ITR core |
| Comparative | ITR-GOI) | expression | expression | component |
| Example | number | cassette | cassette | (ITR-GOI) |
| Comparative | CRI-7 | Cap8 | Rep5 | I-G-1 |
| Example 5 | | | | |
| Example 9 | CRI-8 | Cap8 | RepE | I-G-1 |
| Example 10 | CRI-9 | Cap8 | RepF | I-G-1 |
| Example 11 | CRI-10 | Cap8 | RepG | I-G-1 |
| Example 12 | CRI-11 | Cap8 | RepH | I-G-1 |

### Verification Example 4

The specific steps of the detection of expression of the Rep proteins (Rep78 and Rep52) are as follows.

The above recombinant baculovirus vector DNA was extracted and transfected into Sf9 insect cells to prepare the recombinant baculovirus BEV. The transfected Sf9 insect cells successfully produced BEV, and further infection with a large number of replicating and proliferating BEV caused obvious cytopathic effect (CPE) in Sf9 cells. The culture supernatant of Sf9 cells that had undergone CPE was collected, which contained a large amount of BEV, which was the 0th generation BEV (P0). At the same time, the Sf9 cells containing a large amount of rAAV were collected. The prepared BEV-P0 was infected into suspension-cultured Sf9 cells at a multiplicity of infection (MOI=1). After 72 hours of infection, the cell activity dropped to less than 50%. The cell culture medium was centrifuged at 1000 g for 5 minutes, and the culture supernatant and cell pellet were collected. The supernatant was labeled as the first generation BEV-P1. Continuing to expand the culture, the prepared BEV-PI was infected into suspension-cultured Sf9 cells at a multiplicity of infection (MOI=1). After 72 hours of infection, the cell activity dropped to less than 50%. The cell culture medium was centrifuged at 1000 g for 5 minutes, and the cell pellet was collected for Western Blot to detect the expression of the Rep proteins (Rep78 and Rep52).

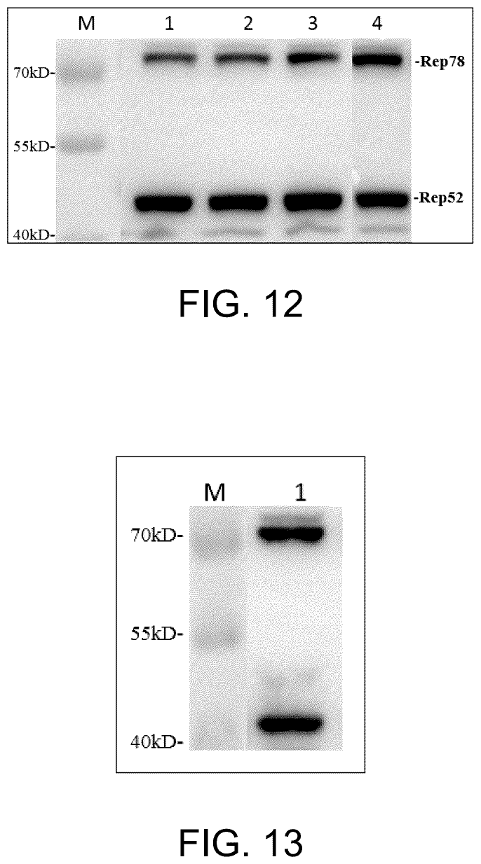

FIGS. 12 to 13 are Western Blot detection diagrams of the Rep proteins (Rep78 and Rep52) of the recombinant baculovirus vector containing the Rep gene expression cassette in Examples 9 to 12 and Comparative Example 5.

FIG. 12 is a Western Blot detection diagram of the Rep proteins (Rep78 and Rep52) of the recombinant baculovirus vectors AcRepE-H containing the Rep gene expression cassette IV, in which lanes 1-4 represent AcRepE-H respectively. It can be seen from FIG. 12 that the above recombinant baculovirus vectors can produce the Rep78 and Rep52 proteins, and the expression amount of the Rep52 protein of AcRepE-G is significantly higher than that of the Rep78 protein. The high stoichiometric ratio of Rep52 and Rep78 proteins may be a key factor in the high yield of rAAV.

FIG. 13 shows the recombinant baculovirus vector AcRep5 containing the Rep gene expression cassette V in Comparative Example 5, in which lane 1 represents the AcRep5 in Comparative Example 5. It can be seen from the figure that the above recombinant baculovirus vectors can produce the Rep78 and Rep52 proteins.

### Verification Example 5

Purification of recombinant AAV virus particles and detection of rAAV virus titer and virus particle solid rate

This verification example uses Q-PCR to detect the titer of the harvested rAAV virus, and the titer unit is expressed in VG/ml (VG, virus genomes). rAAV titers are measured using a pair of primers (Q-ITR-F: GGAACCCCTAGTGATGGAGTT (SEQ ID NO: 26) and Q-ITR-R: CGGCCTCAGTGAGCGA (SEQ ID NO: 27)) targeting the ITR sequence. The specific operation is as follows. The culture was continuously expanded according to step four of Example 9 until the Sf9 cells cultured in the suspension were infected with BEV-P2 seed virus according to the multiplicity of infection (MOI=1) for rAAV packaging. The packaging volume is 300 mL to 400 mL. After 3 days of infection, cell activity was monitored. After the activity was lower than 50%, the cell culture was harvested, 500 μl of the harvested mixed solution (mixed solution of cells and supernatant) was put into a 1.5 ml EP tube, frozen and thawed four times in liquid nitrogen, and treated in a 37° C. water bath. 0.1 μl of Benzonase was added to the 500 μl mixed solution, and the mixed solution was mixed in a 37° C. water bath for 1 hour and then in a 95° C. water bath for 10 minutes. Centrifugation was performed at 2500 g for 10 minutes, and the supernatant was collected in a new 1.5 ml EP tube. 200 μl of the supernatant was added to 10 μl of 10% SDS solution to a final concentration of 0.5% SDS. 1.2 μl Proteinase K (working concentration 112 μg/ml) was then added, the final volume was approximately 210 μl, and a 55° C. water bath was performed for 1 hour. After a short centrifugation, a 95° C. water bath was performed for 10 minutes. After mixing, 5 μl of sample was taken out and 145 μl of ddH₂O was added to make the final volume 150 μl. After mixing, 10 μl of the sample was taken out and 90 μl of ddH₂O was added to make the final volume 100 μl. 2 μl was used as a Q-PCR template to measure the titer of the virus sample. The experimental results are shown in Table 4.

This example uses a technique of analytical ultracentrifugation (abbreviated as AUC) to detect the solid rate (complete capsid: total capsid) of the recombinant adeno-associated virus (rAAV).

Using the sedimentation rate analysis method in the analytical ultracentrifugation technology, the sample is rotated at high speed in the centrifuge, and each component in the sample moves to the bottom of the sample pool. Different components have different movement rates (i.e., sedimentation coefficients) and have different times to fall to the bottom during centrifugation. During this process, the sample distribution state is scanned, and the sedimentation properties of different components are obtained by analyzing its change process over time. The sedimentation coefficient of each component of the sample depends on the molecular weight, molecular shape, and conformation. AUC is generally used to characterize the solid rate of rAAV.

The collected cell culture was purified using an affinity chromatography column (AVIPure AAV8 Resin). 400 μl of the purified rAAV sample was diluted to a dilution factor of OD₂₃₀=0.90, and loaded onto the sample. The sedimentation coefficient and peak area percentage of each peak were recorded. Herein, the sedimentation coefficient of empty shell virus was 55S to 65S, the sedimentation coefficient of solid virus was 80S to 110S, 65S to 80S was the partial part, and greater than 110S was the aggregate. The peaks with sedimentation coefficients between 20S and 150S were normalized to obtain the peak area percentage of each peak, and the empty and solid rate of the virus sample was reported based on the results. The experimental results are shown in Table 4.

TABLE 4
Titer and solid rate detection results of rAAV virions
produced using 5 different recombinant AAV bacmids

| Corresponding | | | rAAV |
|---|---|---|---|
| Example | Recombinant | rAAV | solid rate |
| or Comparative | bacmid AAV | virus titer | (intact capsids: |
| Example | number | (VG/ml) | total capsids) |
| Comparative | CRI-7 | 1.12E+12 | 23% |
| Example 5 | | | |
| Example 9 | CRI-8 | 1.23E+12 | 35% |
| Example 10 | CRI-9 | 1.37E+12 | 39% |
| Example 11 | CRI-10 | 7.37E+11 | 28% |
| Example 12 | CRI-11 | 1.08E+12 | 20% |

As can be seen from Table 4, the titers of rAAV virus particles produced using recombinant AAV bacmids CRI-8 and CRI-9 are higher than those of the control bacmid CRI-7, and the solid rate is significantly higher than that of the control bacmid CRI-7. Compared to the control bacmid CRI-7, the early expression of the Rep78 protein and the higher Rep52/Rep78 ratio in bacmids CRI-8 and CRI-9 may be the key factors for their high titers and the high solid rate. The titer of rAAV virus particles produced using the recombinant AAV bacmid CRI-10 is lower than that of the control bacmid CRI-0, but its solid rate is higher than that of the control bacmid CRI-0. Bacmids CRI-7 and CRI-10 use polH and p6.9 late promoters respectively to drive the expression of the Rep78 protein, where the polH promoter is the most active. Compared to the control bacmid CRI-7, the lower Rep78 protein expression and higher Rep52/Rep78 ratio in the bacmid CRI-10 may be responsible for its relatively low titer and high solid rate. The titer of rAAV virus particles produced using the recombinant AAV bacmid CRI-11 is comparable to that of the control bacmid CRI-0, but its solid rate is slightly lower than that of the control bacmid CRI-7, possibly due to its use of a relatively weak very late promoter to drive Rep78 expression. Increasing the solid rates of viral particles is beneficial to the rAAV product, so fewer particles can be used to obtain a similar amount of genome copies per kilogram, and it is also beneficial to establish a stable downstream purification process.

A person having ordinary skill in the art should be able to easily understand that the above description is only preferred embodiments of the disclosure and is not intended to limit the disclosure. Any modifications, equivalent replacements, and modifications made without departing from the spirit and principles of the disclosure should fall within the protection scope of the disclosure.

The invention claimed is:

1. An expression cassette containing overlapping open reading frames, the overlapping open reading frames being overlapping open reading frames of a first ORF and a second ORF, comprising a second promoter used only to drive gene transcription of the second ORF and further comprising in sequence from a 5′ end to a 3′ end:
a first promoter at least used to drive gene transcription of the first ORF;
a 5′ part of a gene of the first ORF;
an intron; and
a 3′ part of a gene of the second ORF,
wherein the second promoter precedes the 5′ end of the first promoter or within the intron,
wherein the 5′ part of the gene of the first ORF lacks a translation initiation codons ATG of the first ORF, and
wherein during post-transcriptional processing, the translation initiation codons ATG of the first ORF is formed through alternative splicing of the intron.

2. (canceled)

3. The expression cassette according to claim 1, wherein the intron is located between any two nucleotides of the translation initiation codons ATG of the first ORF.

4. The expression cassette according to claim 1, wherein the second promoter is within the intron.

5. The expression cassette according to claim 1, wherein the second promoter precedes the 5′ end of the first promoter.

6. (canceled)

7. The expression cassette according to claim 1, wherein the first promoter is only used to drive gene transcription of the first ORF.

8. The expression cassette according to claim 1, wherein the first promoter is used to drive gene transcription of the first ORF and the second ORF.

9. The expression cassette according to claim 1, wherein the gene of the first ORF is used to express a Rep78 and/or Rep68 protein, and the gene of the second ORF is used to express a Rep52 and/or Rep 40 protein.

10. The expression cassette according to claim 1, wherein the gene of the first ORF is used to express a VP1 protein, and the gene of the second ORF is used to express a VP2 and/or VP3 protein(s).

11. The expression cassette according to claim 1, wherein the first promoter is active before the second promoter.

12. The expression cassette according to claim 11, wherein the first promoter is an immediate early promoter, an early promoter, or a late promoter, and the second promoter is a very late promoter.

13. The expression cassette according to claim 1, wherein the first promoter is deltaIE1, IE1, 39K, or p6.9, and the second promoter is polH or p10.

14. The expression cassette according to claim 1, wherein the first promoter is p10, and the second promoter is polH.

15. A nucleic acid molecule, comprising a Cap gene expression cassette and a Rep gene expression cassette, and the Cap gene expression cassette and/or the Rep gene expression cassette is the expression cassette according to claim 1.

16. The nucleic acid molecule according to claim 15, further comprising an exogenous gene.

17. The nucleic acid molecule according to claim 16, wherein AAV inverted terminal repeats are provided at both ends of the exogenous gene.

18. The nucleic acid molecule according to claim 16, wherein the exogenous gene is a reporter gene, and the reporter gene is at least one of a chloramphenicol acetyltransferase encoding gene, a β-galactosidase encoding gene, a β-glucuronidase encoding gene, a Renilla luciferase encoding gene, an alkaline phosphatase encoding gene, a firefly luciferase encoding gene, a green fluorescent protein encoding gene, and a red fluorescent protein encoding gene.

19. The nucleic acid molecule according to claim 16, wherein the exogenous gene is a gene encoding a drug polypeptide, and the drug polypeptide is at least one of lipoprotein esterase, apolipoprotein, cytokine, interleukin, and interferon.

20. A recombinant baculovirus vector, comprising the expression cassette according to claim 1.

21. The recombinant baculovirus vector according to claim 20, wherein the recombinant baculovirus vector is an insect cell compatible vector.

22. An insect cell, comprising the recombinant baculovirus vector according to claim 20.

23. The insect cell according to claim 22, wherein the insect cell is a Spodoptera frugiperda cell, a Trichopodia cell, a Drosophila cell, or a mosquito cell.
